SEQUENCE LISTING (5 sequences)

SEQ ID NO: 1 - protein, 750 aa, Homo sapiens
MGKSESQMDITDINTPKPKKKQRWTPLEISLSVLVLLLTIIAVTMIALYATYDDGICKSSDCIKSAARLIQNMDATTEPCTDFFKYACGGWLKRNVIPETSSRYGNFDILRDELEVVLKDVLQEPKTEDIVAVQKAKALYRSCINESAIDSRGGEPLLKLLPDIYGWPVATENWEQKYGASWTAEKAIAQLNSKYGKKVLINLFVGTDDKNSVNHVIHIDQPRLGLPSRDYYECTGIYKEACTAYVDFMISVARLIRQEERLPIDENQLALEMNKVMELEKEIANATAKPEDRNDPMLLYNKMTLAQIQNNFSLEINGKPFSWLNFTNEIMSTVNISITNEEDVVVYAPEYLTKLKPILTKYSARDLQNLMSWRFIMDLVSSLSRTYKESRNAFRKALYGTTSETATWRRCANYVNGNMENAVGRLYVEAAFAGESKHVVEDLIAQIREVFIQTLDDLTWMDAETKKRAEEKALAIKERIGYPDDIVSNDNKLNNEYLELNYKEDEYFENIIQNLKFSQSKQLKKLREKVDKDEWISGAAVVNAFYSSGRNQIVFPAGILQPPFFSAQQSNSLNYGGIGMVIGHEITHGFDDNGRNFNKDGDLVDWWTQQSASNFKEQSQCMVYQYGNFSWDLAGGQHLNGINTLGENIADNGGLGQAYRAYQNYIKKNGEEKLLPGLDLNHKQLFFLNFAQVWCGTYRPEYAVNSIKTDVHSPGNFRIIGTLQNSAEFSEAFHCRKNSYMNPEKKCRVW

SEQ ID NO: 2 - protein, 893 aa, Homo sapiens
MATEEFIIRIPPYHYIHVLDQNSNVSRVEVGPKTYIRQDNERVLFAPMRMVTVPPRHYCTVANPVSRDAQGLVLFDVTGQVRLRHADLEIRLAQDPFPLYPGEVLEKDITPLQVVLPNTALHLKALLDFEDKDGDKVVAGDEWLFEGPGTYIPRKEVEVVEIIQATIIRQNQALRLRARKECWDRDGKERVTGEEWLVTTVGAYLPAVFEEVLDLVDAVILTEKTALHLRARRNFRDFRGVSRRTGEEWLVTVQDTEAHVPDVHEEVLGVVPITTLGPHNYCVILDPVGPDGKNQLGQKRVVKGEKSFFLQPGEQLEQGIQDVYVLSEQQGLLLRALQPLEEGEDEEKVSHQAGDHWLIRGPLEYVPSAKVEVVEERQAIPLDENEGIYVQDVKTGKVRAVIGSTYMLTQDEVLWEKELPPGVEELLNKGQDPLADRGEKDTAKSLQPLAPRNKTRVVSYRVPHNAAVQVYDYREKRARVVFGPELVSLGPEEQFTVLSLSAGRPKRPHARRALCLLLGPDFFTDVITIETADHARLQLQLAYNWHFEVNDRKDPQETAKLFSVPDFVGDACKAIASRVRGAVASVTFDDFHKNSARIIRTAVFGFETSEAKGPDGMALPRPRDQAVFPQNGLVVSSVDVQSVEPVDQRTRDALQRSVQLAIEITTNSQEAAAKHEAQRLEQEARGRLERQKILDQSEAEKARKELLELEALSMAVESTGTAKAEAESRAEAARIEGEGSVLQAKLKAQALAIETEAELQRVQKVRELELVYARAQLELEVSKAQQLAEVEVKKFKQMTEAIGPSTIRDLAVAGPEMQVKLLQSLGLKSTLITDGSTPINLFNTAFGLLGMGPEGQPLGRRVASGPSPGEGISPQSAQAPQAPGDNHVVPVLR

SEQ ID NO: 3 - protein, 1280 aa, Homo sapiens
MDLEGDRNGGAKKKNFFKLNNKSEKDKKEKKPTVSVFSMFRYSNWLDKLYMVVGTLAAIIHGAGLPLMMLVFGEMTDIFANAGNLEDLMSNITNRSDINDTGFFMNLEEDMTRYAYYYSGIGAGVLVAAYIQVSFWCLAAGRQIHKIRKQFFHAIMRQEIGWFDVHDVGELNTRLTDDVSKINEGIGDKIGMFFQSMATFFTGFIVGFTRGWKLTLVILAISPVLGLSAAVWAKILSSFTDKELLAYAKAGAVAEEVLAAIRTVIAFGGQKKELERYNKNLEEAKRIGIKKAITANISIGAAFLLIYASYALAFWYGTTLVLSGEYSIGQVLTVFFSVLIGAFSVGQASPSIEAFANARGAAYEIFKIIDNKPSIDSYSKSGHKPDNIKGNLEFRNVHFSYPSRKEVKILKGLNLKVQSGQTVALVGNSGCGKSTTVQLMQRLYDPTEGMVSVDGQDIRTINVRFLREIIGVVSQEPVLFATTIAENIRYGRENVTMDEIEKAVKEANAYDFIMKLPHKFDTLVGERGAQLSGGQKQRIAIARALVRNPKILLLDEATSALDTESEAVVQVALDKARKGRTTIVIAHRLSTVRNADVIAGFDDGVIVEKGNHDELMKEKGIYFKLVTMQTAGNEVELENAADESKSEIDALEMSSNDSRSSLIRKRSTRRSVRGSQAQDRKLSTKEALDESIPPVSFWRIMKLNLTEWPYFVVGVFCAIINGGLQPAFAIIFSKIIGVFTRIDDPETKRQNSNLFSLLFLALGIISFITFFLQGFTFGKAGEILTKRLRYMVFRSMLRQDVSWFDDPKNTTGALTTRLANDAAQVKGAIGSRLAVITQNIANLGTGIIISFIYGWQLTLLLLAIVPIIAIAGVVEMKMLSGQALKDKKELEGAGKIATEAIENFRTVVSLTQEQKFEHMYAQSLQVPYRNSLRKAHIFGITFSFTQAMMYFSYAGCFRFGAYLVAHKLMSFEDVLLVFSAVVFGAMAVGQVSSFAPDYAKAKISAAHIIMIIEKTPLIDSYSTEGLMPNTLEGNVTFGEVVFNYPTRPDIPVLQGLSLEVKKGQTLALVGSSGCGKSTVVQLLERFYDPLAGKVLLDGKEIKRLNVQWLRAHLGIVSQEPILFDCSIAENIAYGDNSRVVSQEEIVRAAKEANIHAFIESLPNKYSTKVGDKGTQLSGGQKQRIAIARALVRQPHILLLDEATSALDTESEKVVQEALDKAREGRTCIVIAHRLSTIQNADLIVVFQNGRVKEHGTHQQLLAQKGIYFSMVSVQAGTKRQ

SEQ ID NO: 4 - protein, 655 aa, Homo sapiens
MSSSNVEVFIPVSQGNTNGFPATASNDLKAFTEGAVLSFHNICYRVKLKSGFLPCRKPVEKEILSNINGIMKPGLNAILGPTGGGKSSLLDVLAARKDPSGLSGDVLINGAPRPANFKCNSGYVVQDDVVMGTLTVRENLQFSAALRLATTMTNHEKNERINRVIQELGLDKVADSKVGTQFIRGVSGGERKRTSIGMELITDPSILFLDEPTTGLDSSTANAVLLLLKRMSKQGRTIIFSIHQPRYSIFKLFDSLTLLASGRLMFHGPAQEALGYFESAGYHCEAYNNPADFFLDIINGDSTAVALNREEDFKATEIIEPSKQDKPLIEKLAEIYVNSSFYKETKAELHQLSGGEKKKKITVFKEISYTTSFCHQLRWVSKRSFKNLLGNPQASIAQIIVTVVLGLVIGAIYFGLKNDSTGIQNRAGVLFFLTTNQCFSSVSAVELFVVEKKLFIHEYISGYYRVSSYFLGKLLSDLLPMRMLPSIIFTCIVYFMLGLKPKADAFFVMMFTLMMVAYSASSMALAIAAGQSVVSVATLLMTICFVFMMIFSGLLVNLTTIASWLSWLQYFSIPRYGFTALQHNEFLGQNFCPGLNATGNNPCNYATCTGEEYLVKQGIDLSPWGLWKNHVALACMIVIFLTIAYLKLLFLKKYS

SEQ ID NO: 5 - protein, 1531 aa, Homo sapiens
MALRGFCSADGSDPLWDWNVTWNTSNPDFTKCFQNTVLVWVPCFYLWACFPFYFLYLSRHDRGYIQMTPLNKTKTALGFLLWIVCWADLFYSFWERSRGIFLAPVFLVSPTLLGITTLLATFLIQLERRKGVQSSGIMLTFWLVALVCALAILRSKIMTALKEDAQVDLFRDITFYVYFSLLLIQLVLSCFSDRSPLFSETIHDPNPCPESSASFLSRITFWWITGLIVRGYRQPLEGSDLWSLNKEDTSEQVVPVLVKNWKKECAKTRKQPVKVVYSSKDPAQPKESSKVDANEEVEALIVKSPQKEWNPSLFKVLYKTFGPYFLMSFFFKAIHDLMMFSGPQILKLLIKFVNDTKAPDWQGYFYTVLLFVTACLQTLVLHQYFHICFVSGMRIKTAVIGAVYRKALVITNSARKSSTVGEIVNLMSVDAQRFMDLATYINMIWSAPLQVILALYLLWLNLGPSVLAGVAVMVLMVPVNAVMAMKTKTYQVAHMKSKDNRIKLMNEILNGIKVLKLYAWELAFKDKVLAIRQEELKVLKKSAYLSAVGTFTWVCTPFLVALCTFAVYVTIDENNILDAQTAFVSLALFNILRFPLNILPMVISSIVQASVSLKRLRIFLSHEELEPDSIERRPVKDGGGTNSITVRNATFTWARSDPPTLNGITFSIPEGALVAVVGQVGCGKSSLLSALLAEMDKVEGHVAIKGSVAYVPQQAWIQNDSLRENILFGCQLEEPYYRSVIQACALLPDLEILPSGDRTEIGEKGVNLSGGQKQRVSLARAVYSNADIYLFDDPLSAVDAHVGKHIFENVIGPKGMLKNKTRILVTHSMSYLPQVDVIIVMSGGKISEMGSYQELLARDGAFAEFLRTYASTEQEQDAEENGVTGVSGPGKEAKQMENGMLVTDSAGKQLQRQLSSSSSYSGDISRHHNSTAELQKAEAKKEETWKLMEADKAQTGQVKLSVYWDYMKAIGLFISFLSIFLFMCNHVSALASNYWLSLWTDDPIVNGTQEHTKVRLSVYGALGISQGIAVFGYSMAVSIGGILASRCLHVDLLHSILRSPMSFFERTPSGNLVNRFSKELDTVDSMIPEVIKMFMGSLFNVIGACIVILLATPIAAIIIPPLGLIYFFVQRFYVASSRQLKRLESVSRSPVYSHFNETLLGVSVIRAFEEQERFIHQSDLKVDENQKAYYPSIVANRWLAVRLECVGNCIVLFAALFAVISRHSLSAGLVGLSVSYSLQVTTYLNWLVRMSSEMETNIVAVERLKEYSETEKEAPWQIQETAPPSSWPQVGRVEFRNYCLRYREDLDFVLRHINVTINGGEKVGIVGRTGAGKSSLTLGLFRINESAEGEIIIDGINIAKIGLHDLRFKITIIPQDPVLFSGSLRMNLDPFSQYSDEEVWTSLELAHLKDFVSALPDKLDHECAEGGENLSVGQRQLVCLARALLRKTKILVLDEATAAVDLETDDLIQSTIRTQFEDCTVLTIAHRLNTIMDYTRVIVLDKGEIQEYGAPSDLLQQRGLFYSMAKDAGLV

(19) United States
(12) Patent Application Publication
(10) Pub. No.: US 2010/0209959 A1
(43) Pub. Date: Aug. 19, 2010

(54) TUMOUR-CELL-FIXING CELLS

(75) Inventors: Massoud Mirshahi, Saint Gratien (FR); Loic Vincent, Evry (FR); Pezhman Mirshahi, Saint Gratien (FR); Jeannette Soria, Taverny (FR); Jean-Pierre Marie, Sevres (FR); Arash Rafii Tabrizi, Cachan (FR)

Correspondence Address: YOUNG & THOMPSON, 209 Madison Street, Suite 500, Alexandria, VA, 22314, US

(73) Assignee: UNIVERSITE PIERRE ET MARIE CURIE-PARIS VI, Paris (FR)

(21) Appl. No.: 12/666,762

(22) PCT Filed: Jun. 27, 2008
(86) PCT No.: PCT/FR08/51188

§ 371 Date: Apr. 8, 2010

(30) Foreign Application Priority Data
Jun. 27, 2007 (FR) 07 56077

(51) Int. Cl. C12Q 1/02; C12N 5/07

(52) U.S. Cl. 435/29; 435/325; 435/395

(57) ABSTRACT

The present invention relates to an isolated cell capable of fixing tumour cells, expressing the CD10 protein and expressing at least one MDR protein, and to the use of this cell for screening for anti-tumour compounds.

The present invention relates to a novel type of cells which fix tumour cells in vivo and in vitro, and to the use of these cells in the screening of compounds for anti-tumour action.

Many cytotoxic molecules capable of killing tumour cells have been identified by the pharmaceutical industry.

Nevertheless, it has been found that, in oncological therapy, the efficacy of the molecules is reduced by a resistance phenomenon. This resistance to anti-tumour agents continues to be a major obstacle to the success of anti-cancer treatments.

Several cellular events have been proposed as resistance factors. They include efflux mechanisms (for example via MDR channels; multi drug resistance), inactivation of the anti-tumour agents (for example by a resistance to antipyrimidic, antimetabolic, etc. agents), mutation of the targets of the anti-tumour agents (for example mutation of the topoisomerase), resistance to apoptosis (for example due to mutation of p53, overexpression of bcl-2, etc.).

It has also been suggested that tumour stem cells would be present in solid tumours. Such tumour stem cells would be more resistant to anti-cancer treatments and would be the origin of resistance and recurrence phenomena (Dean et al. (2005) Nature Rev. Cancer 5:275-284).

No other resistance factor extrinsic to the tumour cell has been identified to date.

The inventors have now succeeded in isolating a novel type of cells which fix and protect tumour cells and which constitute one of the causes of the resistance of tumours to treatments.

### Tumour Cell Fixing Cell

The present invention relates to an isolated cell which is capable of fixing tumour cells, and which expresses the CD10 protein.

This cell which is capable of fixing tumour cells has been named “Hospicell” by the inventors and is also referred to as “fixing cell” or “protecting cell” in the present description.

The ability of an isolated cell according to the invention to fix tumour cells can be demonstrated by numerous techniques which are well known to the person skilled in the art. Mention may thus be made of a method in which (i) cells of the invention are fixed to the walls of a vessel, (ii) tumour cells expressing a fluorescent protein are added to the vessel, (iii) the vessel is washed, and (iv) the fluorescence emitted by the vessel is measured and compared with that emitted by a control vessel to the walls of which cells of the invention have been fixed but to which no tumour cell has been added. The ability to fix tumour cells is proven when the value of the fluorescence measured for the tested cells is greater than that of the cells of the control vessel. Such a method is illustrated in the following Examples. Mention may also be made of methods which use direct observations by optical or electron microscopy, as illustrated in Example 4 and in FIGS. 1 to 3, in which clusters of tumour cells in membrane contact with cells of the invention are observed.

The cells of the invention are capable of fixing tumour cells, such as, for example, leukaemia cells, breast cancer cells or ovarian cancer cells. These cells which are generally large in size (namely about fifty times the size of a tumour cell), are preferably capable of fixing up to 200 tumour cells simultaneously.

The “CD10 protein” (reference in the international classification of enzymes: E.C. 3.4.24.11) is also known by the names Neprilysin, Neutral endopeptidase 24.11, or Common Acute Lymphocytic Leukemia Antigen (CALLA). It is a membrane metallo-peptidase which preferably cleaves polypeptides having fewer than 30 amino acids between basic residues. It has been described as being present at the surface of a limited number of normal or malignant lymphoid progenitors and on some epithelial cells, mainly in the region of the kidney. It is namely described in Shipp et al. (1988) Proc. Natl. Acad. Sci. U.S.A. 85:4819-4823 and under reference P08473 in the UniProtKB database. By way of example, the CD10 protein is represented by SEQ ID NO: 1.

Expression of the CD10 protein may be determined either by detection of its mRNA, or of the RNA precursors thereof, or by detection of the protein itself. Detection of the mRNA of CD10, or of its precursors, can be carried out by various techniques which are well known to the person skilled in the art, such as RT-PCR, for example. Detection of the protein itself can also be carried out by various techniques which are well known to the person skilled in the art. Preferably, detection of the protein itself makes use of specific ligands of CD10, such as antibodies, which can be employed in techniques such as flow cytometry, immunohistochemistry or immunocytochemistry.

Preferably, the level of expression of the CD10 protein is such that it is considered to be strong by the anatomopathologist of the art when it is evaluated, according to the conventional techniques of anatomopathology, on samples included in paraffin with the aid of peroxidase-labelled anti-CD10 antibodies.

In a preferred embodiment of the invention, the cell as defined above expresses an MDR protein.

The expression “MDR protein” denotes a membrane protein which effects active transport of at least one drug and is capable of thus imparting multi-drug resistance to the cell which expresses it. The MDR proteins are notably described in Stavrovskaya (1999) Biochemistry (Moscow) 65:95-106. Preferably, the MDR protein is a ABC type protein which transports drugs from the cytoplasm of the cell which expresses it to the extracellular medium. ABC proteins are characterized in that they comprise at least one adenosine triphosphate (ATP) binding motif called ATP Binding Cassette (ABC) and are well known to the person skilled in the art. ABC type proteins are notably described in Dean et al. (2001) J. Lipid Res. 42:1007-1017 and Szakacs et al. (2006) Nature Reviews Drug Discovery 5:219-234. More preferably, the MDR proteins of ABC type are selected from the proteins of the subfamilies ABCB, ABCC and ABCG.

Preferably, the MDR protein is selected from the group constituted by the LRP protein (Lung Resistance Protein, also called Major Vault Protein (MVP)), the MDR1 protein (MultiDrug Resistance 1, also called ABCB1, or glycoprotein P (Pgp)), the MRP1 protein (Multidrug Resistance-associated Protein 1, also called ABCC1), the MRP2 protein (also called ABCC2), the MRP3 protein (also called ABCC3), the MRP5 protein (also called ABCC5), and the MXR protein (MitoXantrone Resistance protein, also called ABCG2 or Breast Cancer Resistance Protein (BCRP)). More preferably, the MDR protein is selected from the group consisting of the LRP protein, the MDR1 protein, the MRP1 protein, and the MXR protein. Particularly preferably, the cell as defined above expresses the LRP protein and the MDR1 protein at the same time. Yet more preferably, the cell as defined above expresses the LRP protein, the MDR1 protein, the MRP1 protein and the MXR protein at the same time.

The LRP protein is notably represented by SEQ ID NO: 2.

The MDR1 protein is notably represented by SEQ ID NO: 3.

The MXR protein is notably represented by SEQ ID NO: 4.

The MRP1 protein is notably represented by SEQ ID NO: 5.

Expression of the MDR protein can be determined either by detection of its mRNA, or of the RNA precursors thereof, or by detection of the protein itself. Detection of the mRNA of the MDR protein, or of its precursors, can be carried out by various techniques which are well known to the person skilled in the art, such as RT-PCR, for example. Detection of the protein itself can also be carried out by various techniques which are well known to the person skilled in the art. Preferably, detection of the protein itself makes use of specific ligands of the MDR protein, such as antibodies, which can be employed in techniques such as flow cytometry, immunohistochemistry or immunocytochemistry.

When measured by flow cytometry, the level of expression of a MDR protein can be evaluated as follows:

(i) determining the mean fluorescence intensity measured for the cells of the invention with the aid of an antibody directed against the MDR protein;
(ii) determining, under the same conditions as in (i), the mean fluorescence intensity and the fluorescence standard deviation, measured for the cells of the invention with the aid of a control antibody which is of the same isotype as the antibody directed against the MDR protein but does not recognize any antigen of the cells of the invention;
(iii) relating the mean fluorescence intensity determined in (i) to that determined in (ii);
(iv) applying the Kolmorogov-Smirnov test to the ratio determined in (iii).

The Kolmorogov-Smirnov test, which is well known to the person skilled in the art, gives a value, conventionally denoted D, which varies from 0 to 1 and enables measuring a difference between two distributions (here the mean fluorescence intensity measured in (i) and that measured in (ii)). If D=0, the two distributions are superposed; if D=1, the two distributions are completely separate. Two distributions are considered to be significantly distinct when the D value is greater than or equal to 0.15, preferably greater than or equal to 0.2. By way of example, Legrand et al. (2001) Blood 97:502-508 describe the application of the Kolmorogov-Smirnov test to the evaluation of the expression of the Pgp protein.

Accordingly, when measured as indicated above with the aid of the Kolmorogov-Smirnov test, the level of expression of the MDR proteins by the cells of the invention is preferably greater than 0.15, more preferably greater than 0.2.

Preferably, the mean level of expression, by the cells of the invention, given as indicated above with the aid of the Kolmorogov-Smirnov test is:
0.34 for MDR1;
0.57 for MRP1;
0.28 for MRP2;
0.41 for MRP3;
0.70 for MXR; and/or
0.78 for LRP.

Still preferably, the level of expression of the MDR protein is such that it effectively imparts to the cell which expresses it the resistance to a drug that the protein is capable of imparting, when the cell is brought into contact with an amount of drug which is normally toxic for a cell of the same type that does not express the MDR protein. This will be thus herein referred to as the effective level of expression for imparting resistance.

Preferably, the isolated cell as defined above is capable of transferring one or more copies of the MDR protein that it expresses to the tumour cells that it fixes, especially by trogocytosis.

“Trogocytosis” refers to the phenomenon of transfer of molecules from one cell to another which is described especially by Joly & Hudrisier (2003) Nature Immunol. 4:815.

The transfer of MDR protein from an isolated cell of the invention to a tumour cell can be demonstrated in various ways which are well known to the person skilled in the art. By way of example, it is notably possible to carry out an immunodetection of MDR protein expressed by tumour cells before and after they have been brought into contact with cells of the invention.

Preferably, the isolated cell as defined above does not express the following markers: cytokeratin and EMA (Epithelial Membrane Antigen) (specific to epithelial cell lines), vimentin (specific to mesenchymatous cell lines), CD45 (marker of haematopoietic cells, such as granulocytes, monocytes and B and T lymphocytes), CD20 (specific to B lymphocytes), CD3 (specific to T lymphocytes), CD68 (specific to macrophages and histiocytes), CD34 (specific to bone marrow stem cells), S100 protein (specific to melanocytes), myeloperoxidase (specific to polynuclear lines).

In a preferred embodiment of the invention, the isolated cell as defined above is likely to exhibit pseudopods and/or filopods.

“Pseudopods” or “filopods” refer to the evaginations of the cell plasma membrane. Pseudopods can be visualized, for example, by confocal, optical or electron microscopy.

In another preferred embodiment of the invention, the isolated cell as defined above is derived from:
the differentiation of stem cells or of mononucleated cells of bone marrow; or
cells from an effusion of a patient suffering from a cancer.

Preferably, as understood here, the bone marrow stem cells are characterized by the expression of the CD34+ marker and/or the CD133+ marker.

An “effusion” is understood as being a biological fluid which accumulates in a cavity or tissue which does not normally contain any. There may be mentioned by way of example an ascitic fluid, for example from a patient suffering from a ovary or pancreas cancer, or a pleural fluid, for example from a patient suffering from breast cancer.

Preferably, the isolated cell as defined above is not derived from cancer cells taken from an individual.

In another preferred embodiment of the invention, the isolated cell as defined above has been immortalized.

The isolated cell as defined above can be immortalized by any technique known to the person skilled in the art. There may be mentioned by way of example, without implying any limitation, the use of the SV 40 virus T antigen, the use of the EIA region of the adenovirus 2 genome, the use of oncogens such as c-myc or Ha-ras, or the use of human telomerase reverse transcriptase (hTRT) or of a sequence which activates the endogenous hTRT gene.

According to a preferred embodiment of the present invention, immortalization is performed using the SV 40 virus T antigen.

Preferably, the isolated cell as defined above is derived from a cell culture deposited on 20 Jun. 2006 under the Budapest Treaty at the CNCM under number I-3627.

### Obtaining Tumour Cell Fixing Cells

The present invention also relates to a method for obtaining a cell capable of fixing tumour cells, said method comprising the following steps of:
a) cultivating bone marrow mononucleated cells or bone marrow stem cells for a period of time and in a medium suitable for cell differentiation;
b) optionally removing the monocytes from the cells obtained in step a) when the cultivated cells are bone marrow mononucleated cells;
c) incubating the cells obtained in step a) and optionally in step b) with tumour cells, removing the unfixed tumour cells, and recovering the cells to which the tumour cells have become fixed.

The bone marrow mononucleated cells can especially be isolated from bone marrow by Ficoll gradient centrifugation. Before their culture in step a), it is optionally possible to remove the monocytes from the bone marrow mononucleated cells. With regard to bone marrow stem cells, especially CD34+ and/or CD133+, it is, for example, possible to isolate them by immunofixation with the aid of anti-CD34 or anti-CD133 antibodies.

There can be used in step a) a culture medium for stem cells or mononucleated cells of bone marrow which is well known to the person skilled in the art and which contains growth and differentiation factors. This culture is preferably carried out in bottles which have previously been coated with 0.2% gelatin. There may be mentioned as examples, without implying any limitation, the media MV2, HEM (HEPES buffered Eagles medium), DMEM (Dulbecco's modified Eagles medium), GMEM (Glasgow modification of Eagles medium), F-12, etc. Preferably, the medium MV2 (ECBM MV2, Promocell, Heidelberg, Germany) may be used. The growth factors may be selected, without implying any limitation, from fibroblast growth factor (FGF), epidermal growth factor (EGF), insulin-like growth factor (IGF), vascular endothelial growth factor (VEGF), transforming growth factor (TGF-β), stem cell growth factor (SCGF), platelet-derived growth factor (PDGF) or derivatives thereof, combinations of those factors being preferably used. There are optionally added to the culture medium supplements required for cell metabolism, such as amino acids, vitamins such as ascorbic acid, minerals and proteins such as transferrin and derivatives thereof. The culture medium may contain foetal calf serum, chicken serum or equine serum. The culture medium may also contain antibiotics in order to avoid contamination with yeasts, bacteria or fungi, such as penicillin, streptomycin, gentamicin and derivatives thereof. Preferably, after about 6 days' culture, the non-adherent cells are removed and the cells are reincubated in the same medium.

The cells are then cultivated for a period of preferably about 4 weeks, until the cells differentiate into endothelial cells, smooth muscle cells, fibroblasts and other cells, including the desired cells.

In step c), the cells cultivated in step a) are incubated with tumour cells. This step is generally carried out by recovering the adherent cells cultivated in step a) and incubating them with tumour cells suspended in a suitable medium, such as RPMI or DMEM medium. The tumour cells used may be any type of tumour cells. According to a preferred embodiment of the present invention, the tumour cells are HL60 cells (human leukaemia line). The incubation period of the cultivated cells and of the tumour cells varies from about 30 minutes to about 4 hours, preferably about 2 hours. The incubation temperature is preferably 37° C.

In step c), the unfixed tumour cells are generally removed by rinsing with culture medium (optionally the same medium, such as RPMI medium) or with any type of wash solution well known to the person skilled in the art.

The cells to which the tumour cells have become fixed are then recovered, preferably by very brief centrifugation at from 3000 rpm to 6000 rpm.

In an optional subsequent step, the tumour cells are detached from the agglomerate formed by said cell to which residual tumour cells are fixed, by enzymatic or non-enzymatic treatment (trypsin, Accutase® or EDTA 2 mM, followed by very vigorous stirring and repeated washing with a buffer such as PBS).

By way of example to remove the monocytes in step b) or before step a), the mononucleated cells are dispersed on a solid support, such as Petri dishes, or cultured on a solid support (for example gelatin-coated plates) in a suitable medium as defined above. After incubation for about 15 to 30 minutes, the monocytes adhere to the support and the cells that have not adhered yet are collected.

Optionally, in order to confirm that the recovered cells are the fixing cells of interest, they are incubated with tumour cells, preferably HL60 cells (10⁶ HL60 cells/2×10⁵ adherent cells) and the ability of the tumour cells to become fixed to the fixing cells is observed.

The present invention relates also to a method for obtaining a cell capable of fixing tumour cells, said method comprising the following steps of:
a) cultivating cells taken from an effusion of a patient suffering from a cancer;
b) optionally removing the monocytes from the cells obtained in step a);
c) incubating the cells obtained in step a) or b) with tumour cells, removing the unfixed tumour cells, and recovering the cells to which the tumour cells have become fixed; or
d) depositing the cells obtained in step a) or b) on a solid support and recovering the cells that adhere the most rapidly to said support.

Preferably, before being cultured in step a), the cells derived from an effusion are isolated. Still preferably, the cells derived from an effusion are freed of the monocytes prior to being cultured in step a).

Moreover, in step a), the culture medium used may be any cell culture medium known to the person skilled in the art. There may be mentioned by way of example MV2, RPMI, Iscove's MDM or DMEM. The culture medium may optionally contain growth factors, such as those mentioned above, supplements required for cell metabolism, such as amino acids, vitamins such as ascorbic acid, minerals and proteins such as transferrin and their derivatives. The culture medium may optionally contain foetal calf serum, chicken serum or equine serum. The culture medium may also contain antibiotics in order to avoid contamination with yeasts, bacteria or fungi, such as penicillin, streptomycin, gentamicin and their derivatives. Under these conditions, the fixing cells show as early as the first days of culture (1 to 4 days).

Steps b) and c), and the removal of the monocytes prior to step a), may be carried out as described above for the bone marrow mononucleated cells or the bone marrow stem cells.

Preferably, in step d), the cells, previously freed of the monocytes, are deposited on a solid support. The cells that have become fixed to said support are then treated, preferably with Accutase®, and the cells that become detached the most rapidly from the support are recovered. Preferably, the cells that become detached within 5 minutes are recovered.

The support to which the cells become fixed can be any type of solid support well known to the person skilled in the art. As examples of solid supports there may be mentioned glass, plastics, metals, resins or other suitable solid supports to which the cells can be fixed. The term “solid support” also includes materials considered to be semi-solid supports. The solid support can have any suitable form, such as a bead or microparticle, a tube, a Petri dish, a microscope slide, etc.

Optionally, in order to confirm that the recovered cells are the fixing cells of interest, they are incubated with tumour cells, preferably HL60 cells (10⁶ HL60 cells/2×10⁵ adherent cells) and the ability of the tumour cells to become fixed to the fixing cells is observed.

Optionally, at the end of the methods above, the resulting cells can be immortalized, especially as described above.

The invention also relates to a cell likely to be obtained by one of the methods described above.

The cell cultures produced from the isolated cell as defined above, or from the cell likely to be obtained by one of the methods described above, also form part of the invention.

### Screening

The present invention relates to the use of isolated cells as defined above, or of cells likely to be obtained by one of the methods described above, for screening for anti-tumour compounds.

“Anti-tumour compounds” are here understood as being any compound which enables tumour progression to be prevented and/or slowed. In particular, the anti-tumour compounds are compounds that induce or facilitate, directly or indirectly, the death of the tumour cells. More particularly, the anti-tumour compounds according to the invention can induce or facilitate the death of the cells protecting the tumour cells in vivo.

The present invention thus relates to a method of screening for anti-tumour compounds, in which:
isolated cells as defined above, or cells likely to be obtained by one of the methods described above, are contacted with compounds to be screened;
the cell growth and the cell death of the cells contacted with the compounds to be screened are determined;
the compounds that induce a decrease in the cell growth or an increase in the cell death of the cells with which they have been contacted, relative to identical cells that have not been contacted with the compounds to be screened, are selected.

Advantageously, the compounds screened by this method specifically target the cells according to the invention.

The present invention also relates to a method of screening for anti-tumour compounds in which:
a co-culture of isolated cells as defined above, or of cells likely to be obtained by one of the methods described above, and tumour cells is contacted with compounds to be screened;
the cell growth and the cell death of the tumour cells of the co-culture contacted with the compounds to be screened are determined;
the compounds that induce a decrease in the cell growth or an increase in the cell death of the tumour cells of the co-culture, relative to tumour cells in co-culture with isolated cells as defined above, or with cells likely to be obtained by one of the methods described above, that have not been contacted with the compounds to be screened, are selected.

Advantageously, this method permits the selection of the compounds having an anti-tumour action which is not hindered by the resistance to anti-tumour agents provided by the cells of the invention.

The present invention also relates to a method of screening for anti-tumour compounds in which:
compounds to be screened are contacted with isolated cells as defined above, or with cells likely to be obtained by one of the methods described above, and with tumour cells;
the amount of tumour cells fixed by the isolated cells as defined above, or by the cells likely to be obtained by one of the methods described above, is determined;
the compounds that induce a decrease in the amount of tumour cells fixed by the isolated cells as defined above, or by the cells likely to be obtained by one of the methods described above, relative to the amount of tumour cells fixed by the isolated cells as defined above, or by the cells likely to be obtained by one of the methods described above, in the absence of the compounds to be screened, are selected.

This method advantageously permits the selection of the compounds which inhibit the fixing of the tumour cells by the cells of the invention and therefore the protection imparted by the cells of the invention to the tumour cells. These compounds therefore increase the sensitivity of the tumour cells to the anti-tumour compounds.

“Cell death” is understood as being apoptosis, necrosis or any other mechanism that induces the death of the cell. According to a preferred embodiment, the ability of the candidate compound to induce cell death by apoptosis is determined. Any technique well known to the person skilled in the art can be used to measure cell death. The following techniques may be mentioned by way of example, without implying any limitation: labelling with annexin V, use of trypan blue, use of propidium iodide, the TUNEL (Transferase dUTP Nick End Labeling) assay, evaluation of the products of DNA degradation, measurement of caspases (quantitative evaluation and evaluation by activity), etc.

In the above screening methods, the tumour cells used may be any tumour cell. According to a preferred embodiment, the tumour cells are HL60 cells (human leukaemia line) or MDA-MB 231 cells (human breast cancer line) or the patient's own cells (for example ovarian cancer).

The compound to be screened may be any compound of natural or synthetic origin, whether it is already being marketed as a chemotherapeutic agent or is in the course of development or characterization. It may be a mixture of several identified or unidentified molecules, such as for example an extract of animal or plant origin.

The cells according to the invention may readily be employed in high throughput screening protocols (HTS) in order to optimize the current methods of finding candidate compounds that are effective for anti-tumour therapy in general.

In addition, the cells of the invention may also be used for testing the efficacy of candidate compounds for an anti-tumour therapy for a given individual, in order to propose the most appropriate therapy for each individual affected by a cancer. Within this context, it is advantageous to test a panel of chemotherapeutic agents as candidate compounds, with tumour cells taken from the patient himself, in the presence of the fixing cells of the invention.

In a particular embodiment, the fixing cells used in the screening test are taken from the patient himself.

### Diagnostics

The present invention also relates to an in vitro method for diagnosing a cancer, in which the presence of isolated cells as defined above in a sample derived from a tissue suspected of containing a tumour is determined, the presence of isolated cells as defined above being indicative of the presence of a tumour.

Indeed, as has been shown by the inventors, the presence of cells according to the invention in the tissue generally implies the presence of tumour cells close by.

The following examples and figures illustrate the invention without limiting the scope thereof.

### LEGEND OF THE FIGURES

FIGS. 1 and 2 are optical microscopy images (objective 20) of hospicells (obtained by differentiation of CD34+ bone marrow stem cells) which have been contacted with HL60 cells, after incubation for 4 hours (FIG. 1) and 36 hours (FIG. 2).

FIG. 3 is an optical microscopy image (objective 20) of hospicells of the ascitic fluid of a patient suffering from ovarian cancer, after addition of HL60 cells to the fluid.

FIG. 4 is an electron microscopy image of an MDA cell adhering to a hospicell.

FIGS. 5 and 6 are graphs showing the influence of the fixing cells on the sensitivity of HL60 cells to aracytin (AraC) or daunorubicin (DNR) (FIG. 5) and of the fixing cells (“hospicells”) to those drugs (FIG. 6).

FIG. 7 is a graph showing the sensitivity of the fixing cells to various known agents used in anti-tumour therapy.

FIG. 8 shows the amount of fluorescence emitted by OVCAR3 cells (Y-axis, arbitrary units) that express GFP, which cells were cultivated with hospicells (first column), alone (second column), with OVCAR3 cells that do not express GFP (third column), with fibroblasts (fourth column) or with HBMECs (fifth column), in the absence or in the presence of carboplatin and/or paclitaxel.

FIG. 9 shows the amount of fluorescence emitted by OVCAR3 cells (Y-axis, arbitrary units) that express GFP, which cells were cultivated with hospicells (first column), alone (second column), with hospicells in a transwell system (third column), in the absence or in the presence of carboplatin and/or paclitaxel.

### EXAMPLES

### Example 1

### Obtaining Hospicells from Bone Marrow Stem Cells

CD34+ cells were isolated from a normal (or pathological) bone marrow sample by density gradient centrifugation in Ficoll-400. The cells so isolated were distributed in a culture bottle coated with 0.2% gelatin and were cultivated in an MV2 medium (ECBM MV2, Promocell, Heidelberg, Germany) supplemented with amphotericin B 50 ng/ml, gentamicin 50 μg/ml, ascorbic acid 1 μg/ml, human fibroblast growth factor (h-FGF) 10 ng/ml, human epidermal growth factor (h-EGF) 5 ng/ml, Long R3 IGF-1 (insulin-like growth factor) 20 ng/ml, human vascular endothelial cells growth factor (h-VEGF) 10 ng/ml and 5% foetal calf serum.

After 6 days' culture, the non-adherent cells were removed and the adherent cells were cultured for a further 3 weeks in fresh selective medium having the same composition (as detailed above).

The adherent cells so obtained were washed with RPMI and then detached by rapid incubation with Accutase®. The cells so detached were washed with RPMI and then
either resuspended in RPMI containing glutamine and antibiotics and then incubated with immortalized HL60 cells of leukaemic origin. After incubation for 120 minutes at 4° C. with gentle stirring, the cell suspension was centrifuged at 6000 rpm for a few seconds (“centrifugation pulse”), the hospicells that had fixed the HL60 cells being found in the centrifugation pellet. The cells that had settled in the bottom of the tube were resuspended in a culture medium (RPMI+foetal calf serum+glutamine+antibiotics) and distributed in plates coated with 0.2% gelatin.
or placed on a plate 6 well-plate coated with gelatin and containing RPMI medium containing foetal calf serum, glutamine and antibiotics. After incubation in a cell incubator, immortalized HL60 tumour cells of leukaemic origin were added to the wells.

After incubation in the cell incubator, the cells were washed with RPMI, and then Accutase® was added. The cells that become detached within 5 minutes are cells that fix the HL60 cells, while the cells that become detached within 10 minutes and 15 minutes are cells that do not fix the HL60 cells. The cells that become detached within 5 minutes were recovered and were distributed on another 6 well-plate coated with gelatin. HL60 cells were added to one of the wells and, after incubation, the cells were washed and the operation of fixing the HL60 cells was repeated. Finally, Accutase® was added to the wells and the cells that became detached within 5 minutes were recovered and cultivated in complete RPMI medium. The cells obtained under these conditions are cells of the invention or “Hospicells”.

### Example 2

### Obtaining Hospicells from the Ascitic Fluid of a Patient Suffering from Ovarian Cancer

The ascitic fluid of a patient suffering from ovarian cancer was taken with the aid of a biopsy trocar. The mononucleated cells of the ascites were isolated by Ficoll gradient centrifugation. The mononucleated cells so isolated were distributed in the wells of a culture plate. After incubation for 30 minutes, the monocytes adhere to the plastic and the cells that have not yet adhered were collected and placed in a gelatin-coated culture plate. The hospicells were then isolated as described in Example 1.

It was also possible to obtain the hospicells directly from the ascites by placing in a gelatin-coated culture plate a small cluster of cells present in the ascites suspended in RPMI enriched with foetal calf serum, glutamine and antibiotics.

Alternatively, the hospicells were obtained from cell aggregates present in the ascitic fluid obtained from patients suffering from stage III ovarian cancer. Briefly, the ascitic fluid was centrifuged in order to obtain a cell pellet. The cell pellet was freed of the lymphocytes and erythrocytes by Ficoll gradient centrifugation, and then the aggregates of hospicells and ovarian cancer cells were separated by dilution. The hospicells were then detached from the ovarian cancer cells by tryptic digestion.

### Example 3

### Phenotypical Characterization of the Hospicells

It was not possible to observe by immunohistochemistry the expression by the hospicells of the following conventional membrane markers: cytokeratin (antibody KL1, Beckman Coulter) EMA (antibody E29, Dako) (specific to epithelial cell lines), vimentin (antibody V9, Beckman Coulter) (specific to mesenchymatous cell lines), CD45 (antibody 2b11 and PD7/26, Dako) (specific to haematopoietic cells), CD20 (antibody L26, Dako) (specific to B lymphocytes), CD3 (antibody SP7, Neomarkers) (specific to T lymphocytes), CD68 (antibody KP1 and PG-M1, Dako) (specific to macrophages and histiocytes), CD34 (antibody OBend10, Dako) (specific to blood stem cells), S100 protein (polyclonal antibody, Dako) (specific to melanocytes), myeloperoxidase (polyclonal antibody, Dako) (specific to polynuclear lines).

However, labelling of CD9 protein (antibody 56C6, Novocastra) and CD10 protein (Novocastra) was found to be positive.

Briefly, immunohistochemistry was carried out on paraffin sections having a thickness of 4 μm. A technique of preliminary recovery of the antigen based on heating in an EDTA buffer (pH 8) was employed prior to incubation with the antibody. The antibody at the appropriate dilution was incubated for 30 minutes and then revealed with the aid of a streptavidin-biotin complex. The sections were then counterstained with hematoxylin. The technique as a whole was carried out automatically on an Autostainer system (Dako).

### Example 4

### Demonstration of the Fixing of the Cancer Cells to the Hospicells

a) To Hospicells Obtained from Bone Marrow Stem Cells

Hospicells obtained according to the protocol of Example 1 were cultured in RPMI medium in the presence of HL60 leukaemia cells.

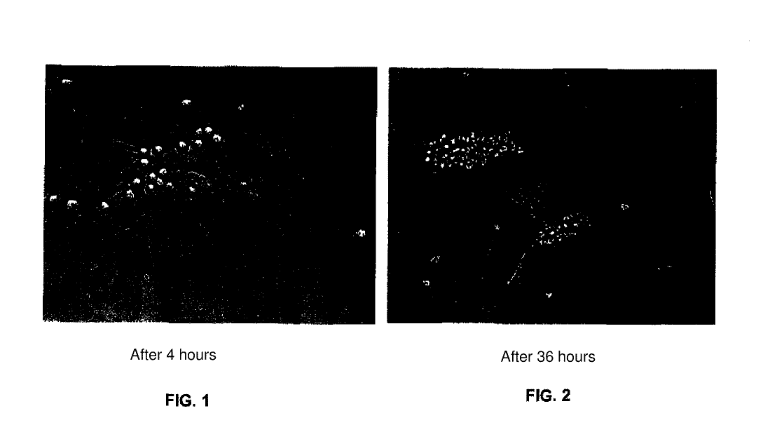

The HL60 cells adhere to the hospicells and form cell clusters in the culture medium. After 4 hours, the fixing of 4 to 8 cells to the fixing cells was observed (FIG. 1). After 36 hours, nodules of malignant cells have formed around the hospicells (FIG. 2).

b) To the Hospicells Present in the Ascitic Fluid of a Patient Suffering from Ovarian Cancer

Ascitic fluid was taken from a patient suffering from ovarian cancer using a biopsy trocar. It was also possible to obtain the hospicells directly from the ascites by placing in a gelatin-coated culture plate a small cluster of cells present in the ascites suspended in RPMI enriched with foetal calf serum, glutamine and antibiotics. The cell clusters of fixing cells already present in the ascitic fluid that have fixed tumour cells were observed by optical microscopy.

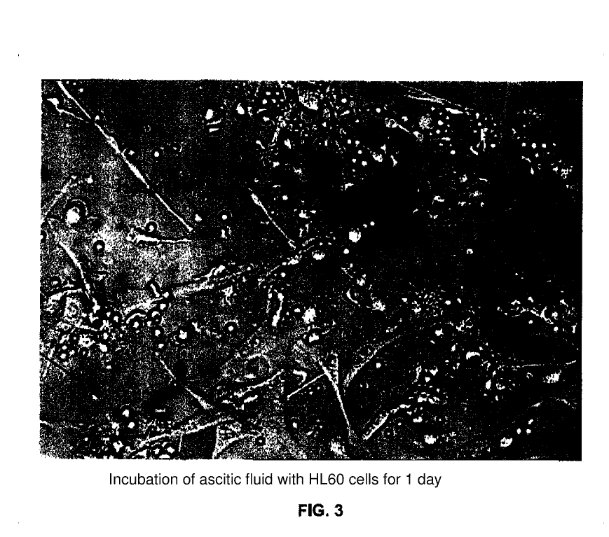

A portion of that fluid was cultured in RPMI medium enriched with foetal calf serum, glutamine and in the presence of HL60 cells for 1 day. FIG. 3 shows the joint fixing of the ovarian cancer cells and the added HL60 cells to the fixing cells.

In addition, the hospicells obtained from cell aggregates of ascitic fluid exhibited particular structures under optical microscopy. Those cells indeed develop long pseudopods, which form a type of “cell thread”. Furthermore, interaction of the hospicells with ovarian cancer cells is visible by confocal microscopy. The hospicells appear therein as cells of large size which are able to establish interactions with several cancer cells at the same time, thus providing a hammock, as it were, for the cancer cells.

Moreover, an immunohistochemical experiment carried out on peritoneal biopsies of patients suffering from ovarian cancer with the aid of the CD10 marker shows the presence of hospicells around cancerous cell aggregates, the hospicells forming a thread around the aggregates.

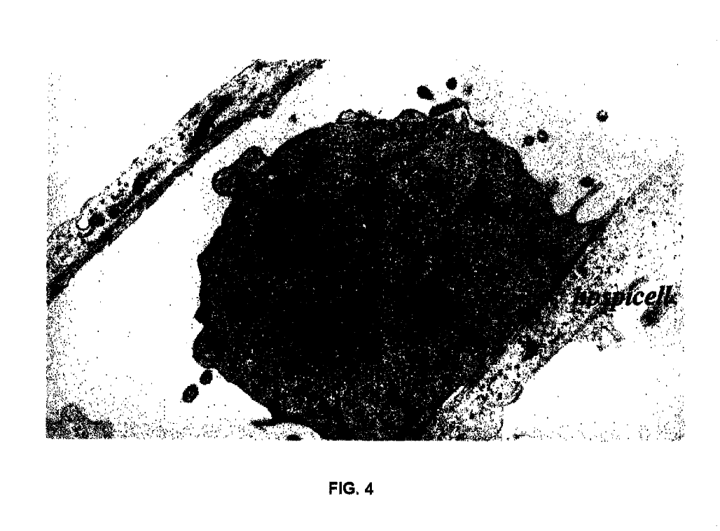

Finally, analysis of the cell aggregates of ascitic fluid by electron microscopy shows that they are formed of hospicells interacting with cancer cells (FIG. 4). It is also possible to observe that the hospicells develop pseudopods, which increases their potential for interaction with cancer cells. More precise observations, carried out on primocultures of hospicells and of cancer cells, showed punctual areas of membrane fusion between the two cell types.

### Example 5

### Interaction Between the Hospicells and the HL60 Cells or the MDA-MB 231 Cells

The inventors have tried to demonstrate the mechanisms of action on which the interaction between the hospicells and the tumour cells is based.

### a) Proteins Involved in the Fixing of HL60

HL60 cells were incubated in RPMI medium under 6 different conditions:
HL60 cells alone (control)
HL60 cells+anti-arrestin antibody (control antibody)
HL60 cells+RGD (Arg-Gly-Asp, integrin consensus sequence, which partially prevents integrin dependent adhesion)
HL60 cells+anti-CD11a antibody
HL60 cells+anti-CD49d antibody
HL60 cells+anti-CD11a antibody+anti-CD49d antibody
HL60 cells+anti-CD11a antibody+anti-CD49d antibody+RGD

For each condition, HL60 cells were deposited separately and in identical amounts in 6 different wells containing complete RPMI medium and fixing cells which had adhered to the support. The ratio between tumour cells and hospicells was 5/1. After incubation for 24 hours at 37° C., the HL60 cells that had not adhered were recovered and counted. The percentage of HL60 cells adhering to the hospicells was then calculated for each condition.

The results show that incubating the HL60 cells beforehand with anti-CD11a or anti-CD49d antibodies or with RGD partially blocks the adhesion of the HL60 cells to the hospicells. Blocking is greater when the HL60 cells have been incubated with the anti-CD11a antibody and the anti-CD49d antibody simultaneously. These results suggest that the CD11a and CD49d integrins are involved in the adhesion between the HL60 cell and the hospicell.

### b) Proteins Involved in the Fixing of MDA

MDA-MB231 cells which had previously been labelled with rhodamine were incubated in complete RPMI medium under 3 different conditions:
MDA cells alone (control)
MDA cells+anti-SDF1 antibody; SDF1 is the ligand of CXCR4 and is a cytokine promoting the formation of breast cancer metastases in the bone marrow
MDA cells+anti-CXCR4 antibody.

MDA cells of each condition were deposited separately and in identical amounts (5 cancer cells per hospicell) in 3 different wells containing RPMI medium and hospicells which had adhered to the support. After incubation for 2 hours at 4° C., the MDA cells that had not adhered were removed, and the amount of cells that had adhered to the hospicells was determined for each condition by measuring the fluorescence with the aid of a plate fluorimeter (Victor fluorimeter).

The results show that incubating the MDA cells beforehand with anti-CXCR4 antibodies induces a marked decrease in the adhesion relative to the control.

These results suggest that CXCR4 modulates the expression of integrins at the surface of MDA tumour cells, which integrins are likely to play a role in the fixing of the tumour cells to the cells of the invention.

### Example 6

### Protective Effect of the Hospicells on the HL60 Cells Against Agents Used in Chemotherapy

Proliferation of HL60 Cells Fixed to Hospicells after Exposure to Aracytin (AraC) or Daunorubicin (DNR)

HL60 cells cultured with hospicells in RPMI medium were treated for 5 days with either aracytin (AraC) or daunorubicin (DNR).

After 5 days' treatment, the drug was removed and the cells were cultured again in fresh medium.

The number of living HL60 cells was measured throughout the experiment using the image analysis system.

The results show that the free HL60 cells die when they are treated with AraC or DNR and that they are no longer detectable when they are fixed to the hospicells. However, after the treatment is stopped, the free HL60 cells do not grow, whereas it is noted that living HL60 cells are identifiable on the hospicells. This effect of “regrowth” of the cells is greater with aracytin than with daunorubicin, the difference being linked to the toxicity of daunorubicin for the hospicells.

These results show that, following treatment with AraC and DNR, the free HL60 cells die, whereas some of the cells bound to the fixing cells remain alive and proliferate after the treatment is stopped.

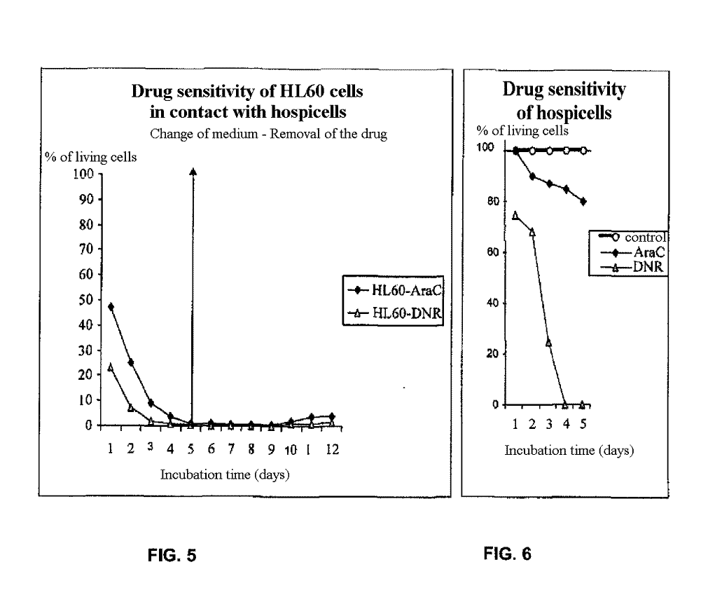

An example of the evaluation of the living cells is shown in FIGS. 5 and 6.

### Example 7

### Sensitivity of Hospicells to Various Known Agents Used in Anti-Tumour Chemotherapy

Immortalized hospicells (M16) were incubated in RPMI medium with adriblastin, bleomycin, deticene, fluorouracil, navelbin, Taxotere or Leustatin for 1 to 3 days.

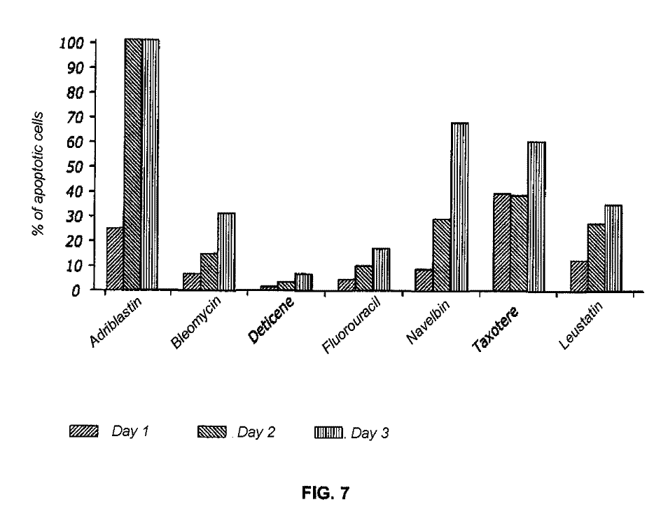

The cells were then detached with Accutase® and the number of cells in apoptosis was detected by annexin V. The percentage of apoptotic cells was then determined by flow cytometry (FIG. 7).

The results show that the hospicells are poorly sensitive to the action of numerous drugs.

### Example 8

### Interaction Between Hospicells and OVCAR3 Cells

The specificity of the interaction of hospicells with cancer cells was then demonstrated with the aid of cells of the ovarian cell line OVCAR3 (Manetta et al. (1988) Eur. J. Gynaecol. Oncol. 9:222-7). An adhesion assay was carried out between hospicells derived from primocultures and OVCAR3 cells expressing GFP (Green Fluorescent Protein). The chosen negative controls represent cell types described in the literature as being microenvironmental cells, namely fibroblasts and bone marrow endothelial cells (HBMEC). In order to be able to distinguish specific hospicell-OVCAR3 cell adhesion from the adhesion of the OVCAR3 cells between themselves, OVCAR3 cells that do not express GFP were also used.

The OVCAR3 cells expressing GFP were obtained by transfection from an RRV virus (Ross River Virus) carrying the VSV-G protein and containing the sequence coding for GFP obtained substantially as described in De Vos et al. (2003) Human Gene Ther. 10:1727-1739. The OVCAR3 cells were spread in culture dishes having a diameter of 35 mm, 24 hours before the transfection. The cells were then transfected with the viruses with a multiplicity of infection of 100:1, the titre of the viral solution having been determined on NIH 3T3 cells as described by Burns et al. (1993) Proc. Natl. Acad. Sci. USA 90:8033-8037. 48 hours after transfection, the expression of GFP was checked by flow cytometry on an FACScan device (Becton Dickinson).

The adhesion assays were conducted as follows. 96-well cell culture dishes containing 0.2% gelatin were covered with hospicells at 70% confluence. The OVCAR3 cells expressing GFP were then seeded at 5·10⁴ cells per well in 200 ml of serum-free medium and were allowed to adhere for 2 hours at 37° C. The non-adherent cells were removed by gentle washing with PBS, and then the amount of adherent cells was determined by measuring the fluorescence of each well with the aid of a Wallac Flite fluorimeter (reading at 560 nm). For each condition, the mean cell density and the standard deviation were calculated from the data obtained for 6 wells. The experiments were repeated 4 times.

The results obtained show that the adhesion of the hospicells is specific to cancer cells. In addition, it is noted that the OVCAR3 cells do not exhibit significant adhesion between themselves, which reinforces the role of the hospicells in the formation of the ascitic aggregates. Finally, the adhesion of the hospicells to the OVCAR3 cells is inhibited by wortmannin, which suggests an active adhesion involving the cytoskeleton.

Secondly, the inventors examined the extent to which the hospicells might impart chemoresistance to the cells to which they bind.

Briefly, the hospicells were cultivated to 60% confluence. 2·10⁴ OVCAR3 cells expressing GFP were then co-cultivated with the hospicells for 24 hours before being brought into contact with a chemotherapeutic agent (carboplatin 22.2 μM and paclitaxel 1.4 μM). The chemotherapeutic effect was determined with the aid of a quantitative colorimetric assay with sulforhodamine B (SRB) as described by Skehan et al. (1990) J. Natl. Cancer Inst. 82:1107-1112. The pink colour of the SRB was quantified by measuring the absorbance at 540 nm. For each condition, the mean cell density and the standard deviation were calculated from the data obtained for 6 wells. The experiments were repeated 3 times.

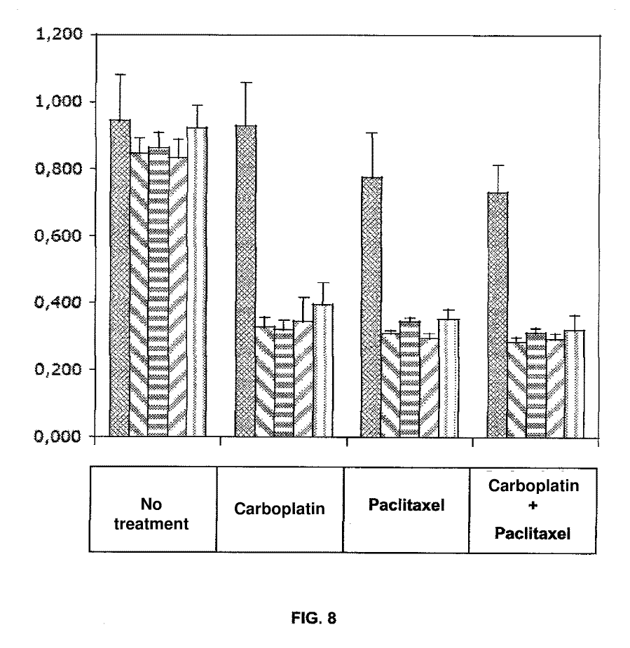

Co-culture of the hospicells and OVCAR3 cells induces chemoresistance with differences of from 2.2 to 2.5 times, according to the therapeutic agent used, in the resistance profile, relative to OVCAR3 cells alone (FIG. 8).

In addition, co-cultures with other control cell types (fibroblasts, HBMEC) do not induce any protection (FIG. 8).

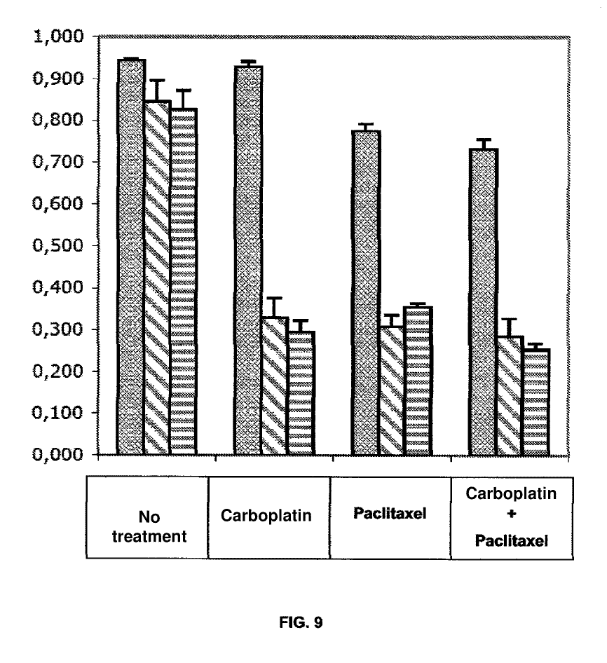

Finally, co-culture experiments in which the culture media of the hospicells and the OVCAR3 cells are able to circulate freely, but contact between hospicells and OVCAR3 cells is excluded (Transwell system), show that direct contact between the cells is necessary for the acquisition of chemoresistance, and that chemoresistance does not pass via any soluble factor (FIG. 9).

### Example 9

### Expression of MDR Proteins by the Hospicells

It is known that the MDR1 protein has been involved in the acquisition, inter alia, of chemoresistance to paclitaxel in tumour cells. Consequently, the expression of MDR proteins by the hospicells was then examined.

The expression of mRNA coding for MDR proteins within the hospicells was first confirmed by RT-PCR. Secondly, the level of expression of the proteins was analyzed by immunofluorescence and flow cytometry.

For the immunofluorescence, the hospicells were deposited on glass coverslides in 6-well plates (Nunc) at a density of 8·10⁴ cells/well in RPMI medium with 10% FCS. 48 hours later, the cells were deprived of serum for 48 hours. The hospicells were then fixed with 3% paraformaldehyde and permeabilized with 0.1% Triton X-100 in PBS. The hospicells were then incubated overnight at 4° C. in a PBS-SAB-Triton 1% mixture with primary antibodies directed against the MDR proteins. Then, a biotinylated secondary antibody and streptavidin associated with fluorescein (Molecular Probes) were used to label the primary antibodies. The antibodies directed against MRP1, MRP2, MRP3, MXR and LRP were supplied by Alexis. The antibodies directed against MDR1 were supplied by Immunotech. The hospicells were then observed under an Axiophot fluorescence microscope (Zeiss) and the images were taken with the aid of a Princeton camera.

For the flow cytometry, the expression of the MDR proteins was determined with the same antibodies as above, with the aid of the Intraprep permeabilization kit (Beckman-Coulter) according to the supplier's instructions, on an EPICS Altra flow cytometry device (Beckman Coulter).

The immunofluorescence results indicate the expression of MDR1, LRP and MXR by the hospicells. Furthermore, the flow cytometry data show that the MDR1, MRP1, MRP2, MRP3, MXR and LRP proteins are expressed by the hospicells (Table 1) with a particularly high level of expression of the MDR1 and LRP proteins, both of which are known to be involved in carboplatin and paclitaxel resistance. The level of expression is determined by relating the mean fluorescence intensity measured with the aid of the anti-MDR antibody to that measured in the presence of an antibody of the same isotype as anti-MDR but not having specificity towards hospicells, then applying the Kolmorogov-Smirnov test (Legrand et al. (2001) Blood 97:502-508).

TABLE 1
Expression of MDR proteins by the hospicells

| | Level of expression of the proteins |
|---|---|
| MDR protein | (Kolmorogov-Smirnov) |
| MDR1 | 0.34 |
| MRP1 | 0.57 |
| MRP2 | 0.28 |
| MRP3 | 0.41 |
| MXR | 0.70 |
| LRP | 0.78 |

These observations were completed by determining the functionality of the MDR proteins expressed by the hospicells by flow cytometry.

This functionality could be established for the MDR1 protein using rhodamine as probe. In addition, the transport of the probe was inhibited by cyclosporin (an MDR1 protein inhibitor). It was also possible to confirm the functionality of the LRP and MXR proteins.

The role of the MDR proteins in the chemoresistance imparted by the hospicells was confirmed by repeating the above co-culture experiments of hospicells and OVCAR3 cells in the presence of carboplatin and paclitaxel, and adding to the culture medium verapamil (1.4 μM), an MDR1 protein inhibitor.

The addition of this inhibitor dramatically reduces the chemoresistance imparted by the hospicells to the OVCAR3 cells, thus demonstrating the involvement of the MDR proteins in this resistance.

In addition, the expression of the MDR1 protein by OVCAR3 cells, cultivated in the presence or in the absence of hospicells, was determined by flow cytometry.

The results obtained show the presence of the MDR1 protein by the OVCAR3 cells. Consequently, the transfer of MDR proteins between the hospicells and the tumour cells is possible. In addition, that transfer also takes place in the presence of a protein translation inhibitor (cycloheximidine A), which suggests an active transfer from the hospicells rather than activation of the translation of those proteins in the tumour cells.

Finally, the inventors have been able to show that the hospicells could transfer membrane fragments to the tumour cells with which they interact.

To that end, the inventors modified the evaluation assay of trogocytosis (active transfer of membrane portions between two cells in close contact) described by Poupot et al. (2003) J. Immunol. 171:2517-2523.

Briefly, the hospicells or the OVCAR3 cells were stained with the green-coloured lipophilic fluorophore PKH67 according to the manufacturer's instructions. The stained cells were then co-cultured with non-stained cells for 0 minutes, 3 minutes and 3 hours, respectively. The co-cultures were conducted in 96 well-culture plates with U-shaped bottoms, with a final concentration of 6×10⁵ cells in 120 μl of complete RPMI 1640 medium supplemented with 10% FCS. The culture plates were then centrifuged for 1 minute at 700 rpm in order to promote contacts between the cells, and were then maintained at 37° C. for one hour in a humid atmosphere containing 5% CO₂. The cells were washed twice in PBS with 0.5 mM EDTA and analyzed by flow cytometry with the aid of an LSRII device and DIVA software (BD Biosciences).

Comparison of the mean fluorescence intensity (mfi) of PKH67 of the unlabelled cells at 0, 3 minutes' or 3 hours' incubation shows an increase consecutive to the co-culture. Accordingly, the cancer cells exhibit an increase in fluorescence after co-culture (mfi from 211 to 3660), which suggests that the cancer cells have acquired membrane fragments from the hospicells.

1-14. (canceled)

15. Isolated cell capable of fixing tumour cells, which cell expresses the CD10 protein and at least one MDR protein.

16. Isolated cell according to claim 15, wherein the MDR protein is selected from the group consisting in the LRP protein (Lung Resistance Protein), the MDR1 protein, the MRP1 protein, the MRP2 protein, the MRP3 protein, the MRP5 protein and the MXR protein.

17. Isolated cell according to claim 15, likely to exhibit or which exhibits pseudopods.

18. Isolated cell according to claim 15, derived from:
the differentiation of stem cells or of mononucleated cells of bone marrow; or
cells of an effusion of a patient suffering from a cancer.

19. Isolated cell according to claim 15, which cell has been immortalized.

20. Isolated cell according to claim 15, derived from a cell culture deposited on 20 Jun. 2006 under the Budapest Treaty at the CNCM under number I-3627.

21. Method for obtaining a cell capable of fixing tumour cells, said method comprising the following steps:
a) cultivating bone marrow mononucleated cells or bone marrow stem cells for a period of time and in a medium suitable for cell differentiation;
b) optionally removing the monocytes from the cells cultivated in step a) when the cultivated cells are bone marrow mononucleated cells;
c) incubating the cells obtained in step a) and optionally in step b) with tumour cells, removing the unfixed tumour cells, and recovering the cells to which the tumour cells have become fixed.

22. Method for obtaining a cell capable of fixing tumour cells, said method comprising the following steps:
a) cultivating cells taken from an ascitic fluid or from a pleural fluid of a patient suffering from a cancer;
b) optionally removing the monocytes from the cells cultivated in step a);
c) incubating the cells obtained in step b) with tumour cells, removing the unfixed tumour cells, and recovering the cells to which the tumour cells have become fixed; or
d) depositing the cells obtained in step b) on a solid support and recovering the cells that adhere the most rapidly to said support.

23. Cell likely to be obtained by a method as defined in claim 21.

24. Method of screening for anti-tumour compounds, wherein isolated cells as defined in claim 15 are used.

25. Method of screening for anti-tumour compounds, which comprises:
contacting isolated cells as defined in claim 15 with compounds to be screened;
determining the cell growth and the cell death of the cells contacted with the compounds to be screened; and
selecting the compounds that induce a decrease in the cell growth or an increase in the cell death of the cells with which they have been contacted, relative to identical cells that have not been contacted with the compounds to be screened.

26. Method of screening for anti-tumour compounds, which comprises:
contacting a co-culture of isolated cells as defined in claim 15 and tumour cells with compounds to be screened;
determining the cell growth and the cell death of the tumour cells of the co-culture contacted with the compounds to be screened; and
selecting the compounds that induce a decrease in the cell growth or an increase in the cell death of the tumour cells of the co-culture, relative to tumour cells in co-culture with the isolated cells that have not been contacted with the compounds to be screened.

27. Method of screening for anti-tumour compounds, which comprises:
contacting compounds to be screened with isolated cells as defined in claim 15, and with tumour cells;
determining the amount of tumour cells fixed by the isolated cells; and
selecting the compounds that induce a decrease in the amount of tumour cells fixed by the isolated cells relative to the amount of tumour cells fixed by the isolated cells, in the absence of the compounds to be screened.

28. In vitro method of diagnosing a cancer, in which the presence of isolated cells as defined in claim 15 in a sample derived from a tissue suspected of containing a tumour is determined, the presence of said isolated cells being indicative of the presence of a tumour.
